(19) United States
(12) Patent Application Publication
(10) Pub. No.: US 2008/0207725 A1
(43) Pub. Date: Aug. 28, 2008

(54) Phenyl-Pyrazole Derivatives as Non-Steroidal Glucocorticoid Receptor Ligands

(75) Inventors: Michael David Barker, Hertfordshire (GB); David House, Hertfordshire (GB); Haydn Terence Jones, Hertfordshire (GB); Simon John Fawcett MacDonald, Hertfordshire (GB); Iain McFarlane Mclay, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, a corporation

(21) Appl. No.: 11/917,775

(22) PCT Filed: Jun. 27, 2006
(86) PCT No.: PCT/EP2006/006246

§ 371 Date: Mar. 14, 2008

(30) Foreign Application Priority Data
Jun. 29, 2005 (GB) 0513297.2

(51) Int. Cl. A61K 31/415; C07D 403/12; C07D 231/38; A61P 29/00; A61P 37/00; A61P 17/00; A61K 9/12; A61K 31/4155

(52) U.S. Cl. 514/407; 548/364.4; 548/371.7; 424/45

(57) ABSTRACT

A compound of formula (I):

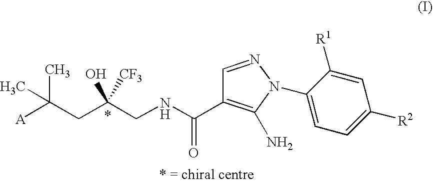

wherein
A represents 2,3-dihydro-1-benzofuran-7-yl, 5-fluoro-2-methoxy-phenyl or 5-fluoro-2-hydroxy-phenyl; and
R¹ and R² each independently represent hydrogen, fluorine or chlorine; or a physiologically functional derivative thereof.

The present invention relates to non-steroidal compounds and a process for their preparation, to pharmaceutical compositions comprising the compounds and the preparation of said compositions, and to use of the compounds for the manufacture of a medicament for therapeutic treatment, particularly for the treatment of inflammation.

Nuclear receptors are a class of structurally related proteins involved in the regulation of gene expression. The steroid hormone receptors are a subset of this family whose natural ligands typically comprise endogenous steroids such as estradiol (estrogen receptor), progesterone (progesterone receptor) and cortisol (glucocorticoid receptor). Man-made ligands to these receptors play an important role in human health, in particular the use of glucocorticoid agonists to treat a wide range of inflammatory conditions.

Glucocorticoids exert their actions at the glucocorticoid receptor (GR) through at least two intracellular mechanisms, transactivation and transrepression (see: Schacke, H., Docke, W-D. & Asadullah, K. (2002) Pharmacol and Therapeutics 96:23-43; Ray, A., Siegel, M. D., Prefontaine, K. E. & Ray, P. (1995) Chest 107:139 S; and Konig, H., Ponta, H., Rahmsdorf, H. J. & Herrlich, P. (1992) EMBO J. 11:2241-2246). Transactivation involves direct binding of the glucocorticoid receptor to distinct deoxyribonucleic acid (DNA) glucocorticoid response elements (GREs) within gene promoters, usually but not always increasing the transcription of the downstream gene product. Recently, it has been shown that the GR can also regulate gene expression through an additional pathway (transrepression) in which the GR does not bind directly to DNA. This mechanism involves interaction of the GR with other transcription factors, in particular NFkB and AP1, leading to inhibition of their pro-transcriptional activity (Schacke, H., Docke, W-D. & Asadullah, K. (2002) Pharmacol and Therapeutics 96:23-43; and Ray, A., Siegel, M. D., Prefontaine, K. E. & Ray, P. (1995) Chest 107:139 S). Many of the genes involved in the inflammatory response are transcriptionally activated through the NFkB and AP1 pathways and therefore inhibition of this pathway by glucocorticoids may explain their anti-inflammatory effect (see: Barnes, P. J. & Adcock, I. (1993) Trend Pharmacol Sci 14:436-441; Cato, A. C. & Wade, E. (1996) Bioessays 18: 371-378).

Despite the effectiveness of glucocorticoids in treating a wide range of conditions, a number of side-effects are associated with pathological increases in endogenous cortisol or the use of exogenous, and particularly systemically administered, glucocorticoids.

These include reduction in bone mineral density (Wong, C. A., Walsh, L. J., Smith, C. J. et al. (2000) Lancet 355:1399-1403), slowing of growth (Allen, D. B. (2000) Allergy 55: suppl 62, 15-18), skin bruising (Pauwels, R. A., Lofdahl, C. G., Latinen, L. A. et al. (1999) N Engl J Med 340:1948-1953), development of cataracts (Cumming, R. G., Mitchell, P. & Leeder, S. R. (1997) N Engl J Med 337:8-14) and dysregulation of lipid and glucose metabolism (Faul, J. L., Tormey, W., Tormey, V. & Burke, C. (1998) BMJ 317:1491; and Andrews, R. C. & Walker, B. R. (1999) Clin Sci 96:513-523). The side-effects are serious enough often to limit the dose of glucocorticoid that can be used to treat the underlying pathology leading to reduced efficacy of treatment.

It has been suggested that excessive activation of the transactivation-GRE pathway may mediate some of these side-effects (see Schacke, H., Docke, W-D. & Asadullah, K. (2002) Pharmacol and Therapeutics 96:23-43). Development of glucocorticoids that selectively modulate the transrepression pathway compared with the transactivation pathway may therefore have a superior anti-inflammatory to side-effect therapeutic index, allowing more effective and safer treatment of the patient. This new class of glucocorticoids could be used to treat more effectively and more safely the whole spectrum of disease currently treated by current glucocorticoids.

Current known glucocorticoids have proved useful in the treatment of inflammation, tissue rejection, auto-immunity, various malignancies, such as leukemias and lymphomas, Cushing's syndrome, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia and Little's syndrome.

Glucocorticoids are especially useful in disease states involving systemic inflammation such as inflammatory bowel disease, systemic lupus erythematosus, polyarteritis nodosa, Wegener's granulomatosis, giant cell arteritis, rheumatoid arthritis, osteoarthritis, seasonal rhinitis, allergic rhinitis, vasomotor rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis and cirrhosis. Glucocorticoids have also been used as immunostimulants and repressors and as wound healing and tissue repair agents.

Glucocorticoids have also found use in the treatment of diseases such as inflammatory scalp alopecia, panniculitis, psoriasis, discoid lupus erythemnatosus, inflamed cysts, atopic dermatitis, pyoderma gangrenosum, pemphigus vulgaris, bullous pemphigoid, systemic lupus erythematosus, dermatomyositis, herpes gestationis, eosinophilic fasciitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitus, erythema nodosum, acne, hirsutism, toxic epidermal necrolysis, erythema multiform and cutaneous T-cell lymphoma.

WO00/32584, WO02/10143, WO03/082827, WO05/003098 and WO05/030213 disclose certain non-steroidal anti-inflammatory agents.

The present invention provides new compounds of formula (I):

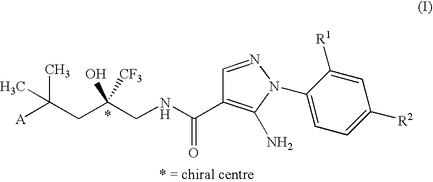

wherein
A represents 2,3-dihydro-1-benzofuran-7-yl, 5-fluoro-2-methoxy-phenyl or 5-fluoro-2-hydroxy-phenyl; and
R¹ and R² each independently represent hydrogen, fluorine or chlorine;
and physiologically functional derivatives thereof (hereinafter “compounds of the invention”).

Compounds of the invention contain one chiral centre and have the 2R configuration as shown in formula (I) above. In one embodiment, the 2R isomer is substantially free of the corresponding 2S isomer. In a further embodiment, the 2R isomer contains less than 5% by weight of the corresponding 2S isomer, such as less than 2% by weight, for example less than 1% by weight.

The term “alkyl” as used herein as a group or a part of a group refers to a straight or branched hydrocarbon chain containing the specified number of carbon atoms. For example, C₁-C₆alkyl means a straight or branched alkyl containing at least 1, and at most 6, carbon atoms. Examples of “alkyl” as used herein include, but are not limited to, methyl, ethyl, n-propyl, n-butyl, n-pentyl, isobutyl, isopropyl, t-butyl and hexyl.

In one embodiment, A represents 2,3-dihydro-1-benzofuran-7-yl. In another embodiment, A represents 5-fluoro-2-methoxy-phenyl. In a further embodiment, A represents 5-fluoro-2-hydroxy-phenyl.

In one embodiment, R¹ represents hydrogen or fluorine and R² represents hydrogen, fluorine or chlorine. In a further embodiment, R¹ and R² each independently represent hydrogen or fluorine. For example, R¹ is hydrogen and R² is fluorine.

In one embodiment, the compound of formula (I) is:
(2R)-5-amino-N-[4-(2,3-dihydro-1-benzofuran-7-yl)-2-hydroxy-4-methyl-2-(trifluoromethyl)pentyl]-1-phenyl-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-(2-fluorophenyl)-1H-pyrazole-4-carboxamide;
(2R)-5-amino-1-(2,4-difluorophenyl)-N-[4-[5-fluoro-2-(methyloxy)phenyl]-2-hydroxy-4-methyl-2-(trifluoromethyl)pentyl]-1H-pyrazole-4-carboxamide;
(2R)-5-amino-1-(4-chlorophenyl)-N-[4-[5-fluoro-2-(methyloxy)phenyl]-2-hydroxy-4-methyl-2-(trifluoromethyl)pentyl]-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-phenyl-1H-pyrazole-4-carboxamide;
(2R)-5-amino-N-[4-(5-fluoro-2-hydroxyphenyl)-2-hydroxy-4-methyl-2-(trifluoromethyl)pentyl]-1-(4-fluorophenyl)-1H-pyrazole-4-carboxamide;
(2R)-5-amino-N-[4-(5-fluoro-2-hydroxyphenyl)-2-hydroxy-4-methyl-2-(trifluoromethyl)pentyl]-1-(2-fluorophenyl)-1H-pyrazole-4-carboxamide;
(2R)-5-amino-1-(2,4-difluorophenyl)-N-[4-(5-fluoro-2-hydroxyphenyl)-2-hydroxy-4-methyl-2-(trifluoromethyl)pentyl]-1H-pyrazole-4-carboxamide;
(2R)-5-amino-1-(4-chlorophenyl)-N-[4-(5-fluoro-2-hydroxyphenyl)-2-hydroxy-4-methyl-2-(trifluoromethyl)pentyl]-1H-pyrazole-4-carboxamide;
(2R)-5-amino-N-[4-(5-fluoro-2-hydroxyphenyl)-2-hydroxy-4-methyl-2-(trifluoromethyl)pentyl]-1-phenyl-1H-pyrazole-4-carboxamide;
(2R)-5-amino-N-[4-(2,3-dihydro-1-benzofuran-7-yl)-2-hydroxy-4-methyl-2-(trifluoromethyl)pentyl]-1-(2-fluorophenyl)-1H-pyrazole-4-carboxamide;
(2R)-5-amino-N-[4-(2,3-dihydro-1-benzofuran-7-yl)-2-hydroxy-4-methyl-2-(trifluoromethyl)pentyl]-1-(4-fluorophenyl)-1H-pyrazole-4-carboxamide;
(2R)-5-amino-1-(2,4-difluorophenyl)-N-[4-(2,3-dihydro-1-benzofuran-7-yl)-2-hydroxy-4-methyl-2-(trifluoromethyl)pentyl]-1H-pyrazole-4-carboxamide; or
(2R)-5-amino-1-(4-chlorophenyl)-N-[4-(2,3-dihydro-1-benzofuran-7-yl)-2-hydroxy-4-methyl-2-(trifluoromethyl)pentyl]-1H-pyrazole-4-carboxamide; or
   a physiologically functional derivative thereof.

The compounds of the invention bind to the glucocorticoid receptor, appear to have glucocorticoid receptor agonist activity and may show improved selectivity for the glucocorticoid receptor over the progesterone receptor and/or may possess advantageous selectivity in respect of maintaining transrepression activity whilst reducing the transactivation activity thereby providing anti-inflammatory properties with fewer or less severe related side effects.

The invention includes physiologically functional derivatives of the compound of formula (I). By the term “physiologically functional derivative” is meant a chemical derivative of a compound of formula (I) having the same physiological function as a free compound of formula (I), for example, by being convertible in the body thereto and includes any pharmaceutically acceptable esters, carbonates, carbamates, salts and solvates of compounds of formula (I), and solvates of any pharmaceutically acceptable esters, carbonates, carbamates or salts of compounds of formula (I), which, upon administration to the recipient, are capable of providing (directly or indirectly) compounds of formula (I) or active metabolite or residue thereof. Thus one embodiment of the invention embraces compounds of formula (I) and salts and solvates thereof. Another embodiment of the invention embraces compounds of formula (I) and salts thereof. A further embodiment of the invention embraces compounds of formula (I).

Salts and solvates of the compounds of formula (I) and physiologically functional derivatives thereof which are suitable for use in medicine are those wherein the counter-ion or associated solvent is pharmaceutically acceptable. However, salts and solvates having non-pharmaceutically acceptable counter-ions or associated solvents are within the scope of the present invention, for example, for use as intermediates in the preparation of other compounds of formula (I) and their pharmaceutically acceptable salts, solvates, and physiologically functional derivatives.

Suitable salts according to the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Examples of solvates include hydrates.

The compounds of the invention are expected to have potentially beneficial anti-inflammatory or anti-allergic effects, particularly upon topical administration, demonstrated by, for example, their ability to bind to the glucocorticoid receptor and to illicit a response via that receptor. Hence, the compounds of the invention may be of use in the treatment of inflammatory and/or allergic disorders.

Examples of disease states in which the compounds of the invention are expected to have utility include skin diseases such as eczema, psoriasis, allergic dermatitis, neurodermatitis, pruritis, exfoliative dermatitis, pemphigus and hypersensitivity reactions; inflammatory conditions of the nose, throat or lungs such as asthma (including allergen-induced asthmatic reactions), rhinitis (including hayfever), nasal polyps, chronic obstructive pulmonary disease (COPD), interstitial lung disease, and fibrosis; inflammatory bowel conditions such as ulcerative colitis and Crohn's disease; auto-immune diseases such as rheumatoid arthritis, systemic lupus erythematosus, termporal arteritis, polyarteritis nodosa, polymyositis, ankylosing spondylitis, sarcoidosis, autoimmune hepatitis; cancers such as acute and lymphatic leukaemia, myeloma, lymphoma; nephritic syndrome; septic shock; adrenal insufficiency; ophthalmic inflammation and allergic conjunctivitis; obesity; diabetes; chronic inflammatory pain including musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea; psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions including, but not limited to, myocardial infarction, diabetes, miscarriage or abortion, etc), anxiety disorders (including generalised anxiety disorder and social anxiety disorder), panic disorder, agoraphobia, social phobia, obsessive compulsive disorder and post-traumatic stress disorder, memory disorders, including dementia, amnesic disorders and age-associated memory impairment, disorders of eating behaviours, including anorexia nervosa and bulimia nervosa, sleep disorders (including disturbances of circadian rhythm, dyssomnia, insomnia, sleep apnea and narcolepsy), withdrawal from abuse of drugs such as of cocaine, ethanol, nicotine, benzodiazepines, alcohol, caffeine, phencyclidine (phencyclidine-like compounds), opiates (e.g. cannabis, heroin, morphine), amphetamine or amphetamine-related drugs (e.g. dextroamphetamine, methylamphetamine) or a combination thereof. Compounds of the invention may also have utility in inducing suppression of the immune system during organ transplantation, in acute transplant reject, angioedema of the upper respiratory tract and anaphylactic shock.

For example, disease states in which the compounds of the invention are expected to have utility include skin diseases such as eczema, psoriasis, allergic dermatitis, neurodermatitis, pruritis and hypersensitivity reactions; inflammatory conditions of the nose, throat or lungs such as asthma (including allergen-induced asthmatic reactions), rhinitis (including hayfever), nasal polyps, chronic obstructive pulmonary disease (COPD), interstitial lung disease, and fibrosis; inflammatory bowel conditions such as ulcerative colitis and Crohn's disease; and auto-immune diseases such as rheumatoid arthritis.

It will be appreciated by those skilled in the art that reference herein to treatment extends to prophylaxis as well as the treatment of established conditions.

As mentioned above, compounds of the invention are expected to be of use in human or veterinary medicine, in particular as anti-inflammatory and/or anti-allergic agents.

There is thus provided as a further aspect of the invention a compound of the invention for use in human or veterinary medicine, particularly in the treatment of patients with inflammatory and/or allergic conditions, such as rheumatoid arthritis, asthma, COPD, allergy and/or rhinitis.

In another aspect of the invention there is provided a compound of the invention, for use in the treatment of patients with skin disease such as eczema, psoriasis, allergic dermatitis, neurodermatitis, pruritis and/or hypersensitivity reactions.

According to another aspect of the invention, there is provided the use of a compound of the invention for the manufacture of a medicament for the treatment of patients with inflammatory and/or allergic conditions, such as rheumatoid arthritis, asthma, COPD, allergy and/or rhinitis.

According to yet to another aspect of the invention, there is provided the use of a compound of the invention for the manufacture of a medicament for the treatment of patients with skin disease such as eczema, psoriasis, allergic dermatitis, neurodermatitis, pruritis and/or hypersensitivity reactions.

In a further or alternative aspect, there is provided a method for the treatment of a human or animal subject with an inflammatory and/or allergic condition, such as rheumatoid arthritis, asthma, COPD, allergy and/or rhinitis, which method comprises administering to said human or animal subject an effective amount of a compound of the invention.

In yet a further or alternative aspect there is provided a method for the treatment of a human or animal subject with skin disease such as eczema, psoriasis, allergic dermatitis, neurodermatitis, pruritis and/or hypersensitivity reactions, which method comprises administering to said human or animal subject an effective amount of a compound of the invention.

The compounds of the invention may be formulated for administration in any convenient way, and the invention therefore also includes within its scope pharmaceutical compositions comprising a compound of the invention together, if desirable, in admixture with one or more physiologically acceptable diluents or carriers.

Further, there is provided a process for the preparation of such pharmaceutical compositions which comprises mixing the ingredients.

The compounds of the invention may, for example, be formulated for oral, nasal, buccal, sublingual, parenteral, local rectal administration or other local administration.

Local administration as used herein, includes administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (e.g. eye or nose drops), solutions/suspensions for nebulisation, suppositories, pessaries, retention enemas and chewable or suckable tablets or pellets (e.g. for the treatment of aphthous ulcers) or liposome or microencapsulation preparations.

Formulations for administration topically to the nose for example, for the treatment of rhinitis, include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of the invention may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Spray compositions may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain a compound of formula (I) and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants eg. oleic acid, lecithin or an oligolactic acid or derivative eg. as described in WO94/21229 and WO98/34596 and cosolvents eg. ethanol.

There is thus provided as a further aspect of the invention a pharmaceutical aerosol formulation comprising a compound of the invention and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of the invention and a suitable powder base such as lactose or starch. Each capsule or cartridge may generally contain between 20%1 g to 10 mg of the compound of the invention. Alternatively, the compound of the invention may be presented without excipients such as lactose.

The proportion of the active compound of the invention in the local compositions according to the invention depends on the precise type of formulation to be prepared but will generally be within the range of from 0.001 to 10% by weight. Generally, for most types of preparations, the proportion used will be within the range of from 0.005 to 1% and preferably from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg preferably from 20 μg to 2000 μg, more preferably from 20 μg to 500 μg of a compound of formula (I). Administration may be once daily or several times daily, for example 2, 3, 4 or 8 times, giving for example 1, 2 or 3 doses each time. The overall daily dose with an aerosol will be within the range of from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and, in particular, in the range of from 1 to 10 microns, such as from 1 to 5 microns, more preferably from 2 to 3 microns.

The formulations of the invention may be prepared by dispersal or dissolution of the medicament and a compound of the invention in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g., see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, most preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak pic, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations, a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Topical preparations may be administered by one or more applications per day to the affected area; over skin areas occlusive dressings may advantageously be used. Continuous or prolonged delivery may be achieved by an adhesive reservoir system.

For internal administration the compounds of the invention may, for example, be formulated in conventional manner for oral, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms are, however, preferred as described below.

The compounds of the invention may in general be given by internal administration in cases wherein systemic glucocorticoid receptor agonist therapy is indicated.

Slow release or enteric coated formulations may be advantageous, particularly for the treatment of inflammatory bowel disorders.

In some embodiments, the compounds of the invention will be formulated for oral administration. In other embodiments, the compounds of the invention will be formulated for inhaled administration. In further embodiments, the compounds of the invention will be formulated for intranasal administration.

The compounds and pharmaceutical formulations according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or antihistamines. The invention thus provides, in a further aspect, a combination comprising a compound of the invention together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or an antihistamine. Suitable combinations include combinations comprising a compound of the invention together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of the invention together with a β₂-adrenoreceptor agonist.

Examples of 2-adrenoreceptor agonists include salmeterol (which may be a racemate or a single enantiomer, such as the R-enantiomer), salbutamol (which may be a racemate or a single enantiomer, such as the R-enantiomer), formoterol (which may be a racemate or a single diastereomer, such as the R,R-diastereomer), salmefamol, fenoterol, carmoterol, etanterol, naminterol, clenbuterol, pirbuterol, flerobuterol, reproterol, bambuterol, indacaterol or terbutaline and salts thereof, for example the xinafoate (1-hydroxy-2-naphthalenecarboxylate) salt of salmeterol, the sulphate salt or free base of salbutamol or the fumarate salt of formoterol. In one embodiment, the β₂-adrenoreceptor agonists are long-acting β₂-adrenoreceptor agonists, for example those having a therapeutic effect over a 24 hour period.

Examples of long acting 2-adrenoreceptor agonists may include those described in WO02/66422A, WO02/270490, WO02/076933, WO03/024439, WO03/072539, WO 03/091204, WO04/016578, WO04/022547, WO04/037807, WO04/037773, WO04/037768, WO04/039762, WO04/039766, WO01/42193 and WO03/042160.

Examples of long-acting β₂-adrenoreceptor agonists include:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl)oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2-(1H)-quinolinon-5-yl)ethylamine; and
5-[(R)-2-(2-[(4-[4-(2-amino-2-methyl-propoxy)-phenylamino]-phenyl]-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Examples of anti-inflammatory agents include non-steroidal anti-inflammatory drugs (NSAID's). Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples CCR3 inhibitors include those disclosed in WO02/26722. Adenosine 2a agonists include those disclosed in WO05/116037

In one embodiment the invention provides the use of the compounds of the invention in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Another compound is cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P.98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (PCT/EP2003/014867) (Glaxo Group Ltd), WO04/056823 (PCT/EP2004/005494) (Glaxo Group Ltd) and WO04/103998 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (for example, CAS 28797-61-7), darifenacin (for example, CAS 133099-04-4, or CAS 133099-07-7 for the hydrobromide sold under the name Enablex), oxybutynin (for example, CAS 5633-20-5, sold under the name Ditropan), terodiline (for example, CAS 15793-40-5), tolterodine (for example, CAS 124937-51-5, or CAS 124937-52-6 for the tartrate, sold under the name Detrol), otilonium (for example, as the bromide, CAS 26095-59-0, sold under the name Spasmomen), trospium chloride (for example, CAS 10405-02-4) and solifenacin (for example, CAS 242478-37-1, or CAS 242478-38-2, or the succinate also known as YM-905 and sold under the name Vesicare).

Other anticholinergic agents include compounds of formula (XXI), which are disclosed in U.S. patent application 60/487,981:

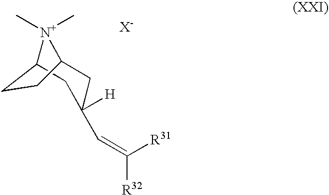

in which the preferred orientation of the alkyl chain attached to the tropane ring is endo; R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having from 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;

X⁻ represents an anion associated with the positive charge of the N atom. X⁻ may be but is not limited to chloride, bromide, iodide, sulfate, benzene sulfonate, and toluene sulfonate, including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds of formula (XXII) or (XXIII), which are disclosed in U.S. patent application 60/511,009:

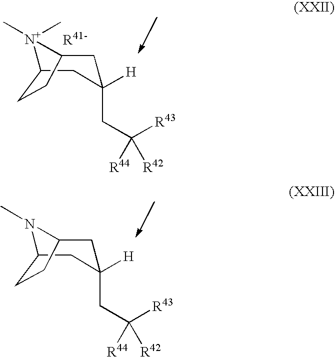

wherein:
the H atom indicated is in the exo position;
R⁴⁻ represents an anion associated with the positive charge of the N atom; R⁴¹⁻ may be but is not limited to chloride, bromide, iodide, sulfate, benzene sulfonate and toluene sulfonate;
R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having from 6 to 10 carbon atoms), heterocycloalkyl (having from 5 to 6 carbon atoms) and N or O as the heteroatom, heterocycloalkyl-alkyl (having from 6 to 10 carbon atoms) and N or O as the heteroatom, aryl, optionally substituted aryl, heteroaryl, and optionally substituted heteroaryl;
R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), —CH₂N(R⁴⁸)CONH(R⁴⁷);
R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;
R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;
R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl, including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

Examples of antihistamines (also referred to as H1-receptor antagonists) include any one or more of the numerous antagonists known which inhibit H1-receptors, and are safe for human use. First generation antagonists, include derivatives of ethanolamines, ethylenediamines, and alkylamines, such as diphenylhydramine, pyrilamine, clemastine, chlorpheniramine. Second generation antagonists, which are non-sedating, include loratidine, desloratidine, terfenadine, astemizole, acrivastine, azelastine, levocetirizine fexofenadine and cetirizine.

Examples of anti-histamines include loratidine, desloratidine, fexofenadine, cetirizine, levocabastine, olopatadine, amlexanox and epinastine.

In one embodiment the invention provides a combination comprising a compound of formula (I) or a pharmaceutically acceptable salt thereof together with an H1 antagonist. Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I), or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

The invention thus provides, in a further aspect a pharmaceutical composition which further comprises another therapeutically active agent.

The invention thus provides, in a further aspect, a pharmaceutical composition in which said therapeutically active agent is a β₂-adrenoreceptor agonist.

The invention thus provides, in a further aspect, a pharmaceutical composition comprising a combination of a compound of the invention together with a PDE4 inhibitor.

The invention thus provides, in a further aspect, a combination comprising a compound of the invention together with a PDE4 inhibitor.

The invention thus provides, in a further aspect, a combination comprising a compound of the invention together with a β₂-adrenoreceptor agonist.

The invention thus provides, in a further aspect, a combination comprising a compound of the invention together with an anticholinergic.

The invention thus provides, in a further aspect, a combination comprising a compound of the invention together with an antihistamine.

The invention thus provides, in a further aspect, a combination comprising a compound of the invention together with a PDE4 inhibitor and a β₂-adrenoreceptor agonist.

The invention thus provides, in a further aspect, a combination comprising a compound of the invention together with an anticholinergic and a PDE-4 inhibitor.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. The individual compounds may be administered simultaneously in a combined pharmaceutical formulation. Appropriate doses of known therapeutic agents will be readily appreciated by those skilled in the art.

A process according to the invention for the preparation of compounds of formula (I) comprises coupling of a carboxylic acid of formula (II):

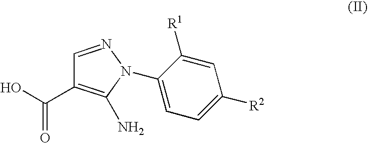

or an activated derivative thereof
wherein the groups R¹ and R² are as defined above for compounds of formula (I),
with an amine of formula (III):

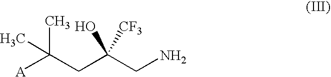

or a protected derivative thereof
wherein the group A is as defined above for compounds of formula (I).

The compound of formula (II) may be activated if necessary, for example, as an acid halide or anhydride. In one embodiment, the compound of formula (III) is not protected.

The coupling may be performed in an inert solvent such as dichloromethane, dimethylformamide, acetonitrile or tetrahydrofuran at a non-extreme temperature from 0 to reflux or 80° C. (whichever is lower) using a standard amide coupling reagent such as those described in “Advanced Organic Chemistry” 5^{th} edition, M. B. Smith and J. March, Wiley, 2001, pp 508-510 and “Comprehensive Organic Transformations” R. C. Larock, VCH, 1989, pp 972-976. In one embodiment, the reaction is performed with dimethylformamide as solvent in the presence of triethylamine or diisopropylethylamine as base using O-(7-azabenzotriazol-1-yl)-N,N,N′,N′-tetramethyluronium hexafluorophosphate as coupling agent at room temperature.

Compounds of formula (III) wherein A represents 2,3-dihydro-1-benzofuran-7-yl or 5-fluoro-2-methoxy-phenyl have been described in racemic form (WO03/082827, WO04/063163). Individual enantiomers of compounds of formula (III) may be obtained, for example, by separation by HPLC on a chiral column of the racemic material (III) or a protected version (IV) thereof.

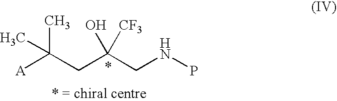

wherein the group A is as defined above for compounds of formula (I), and P represents a protecting group which is removed following enantiomer separation.

In one embodiment, P represents a benzyloxycarbonyl (CBZ), or benzyl protecting group. However, those skilled in the art could envisage the use of other protecting groups as alternatives. The CBZ or benzyl protecting groups may be removed by, for example, hydrogenolysis over a suitable catalyst such as palladium on carbon.

Where this protecting group P in compound (IV) contains an additional chiral centre of defined stereochemistry, for example, in the (R)-1-phenylethylamine derivative (V)

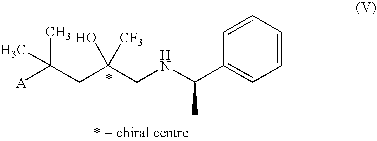

the resulting diastereoisomers may be separated by chromatography on a non-chiral support. As before, deprotection by hydrogenolysis following isomer separation provides the single enantiomers of compound (III).

Compounds of formula (IV) may be prepared directly by protection of the racemic amine (III). Alternatively intermediates of formula (IV) and (V) may be prepared by the reaction of the epoxide (VI)

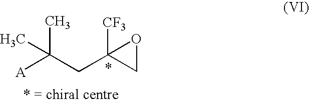

with an amine P—NH₂.
wherein the group A is as defined above for compounds of formula (I).

The epoxide opening reaction may be performed in a dipolar aprotic solvent such as N,N-dimethylformamide at a non-extreme temperature in the range 0-100° C., most commonly 20° C. (or room temperature) in the presence of a strong base such as potassium tert-butoxide. Alternatively, these epoxide opening reactions may be performed in a microwave reactor in the absence of solvent or with a small amount of a high boiling point non-nucleophilic solvent such as N-methylpyrrolidinone at a high temperature in the range 100-200° C., most commonly 150° C. For reactions with (R)-(+)-1-phenylethylamine the epoxide opening to give (V) may be conveniently performed in ethanol solution at 50° C.

Compounds of formula (VI) in which A represents 2,3-dihydro-1-benzofuran-7-yl or 5-fluoro-2-methoxy-phenyl are described in WO04/063163. The compound of formula (VI) in which A represents 5-fluoro-2-methoxy-phenyl has also been described as separate enantiomers in WO 05/234250, WO05/040145 and in Bioorg. Med. Chem. Letters. 2006, 16, 654-657.

Compounds of formula (I) in which A represents 5-fluoro-2-hydroxy-phenyl may alternatively be directly prepared by reaction of the compounds of formula (I) in which A represents 5-fluoro-2-methoxy-phenyl with, for example, boron tribromide in dichloromethane solution.

Compounds of formula (II) are either known in the literature or may be prepared by reaction of a compound of formula (VII):

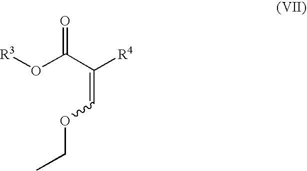

wherein R⁴ represents the group:

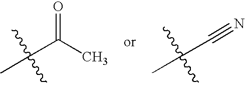

and R³ represents C₁-C₆ alkyl, with an aryl hydrazine of formula (VIII):

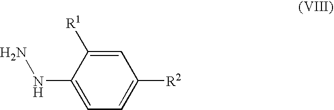

wherein the groups R¹ and R² are as defined for the compounds of formula (I), followed by deprotection of the ester function to the acid function. In one embodiment, R³ represents ethyl.

Coupling may conveniently be carried out under reflux in ethanol. Alternatively, coupling may be achieved by reaction of a compound of formula (VII) with a salt of the compound of formula (VIII), for example the hydrochloride salt, by refluxing in ethanol in the presence of a base such as triethylamine or diisopropylamine.

Subsequent deprotection of the acid group to give the compounds of formula (II) may be effected by refluxing in ethanol in the presence of a strong base such as sodium hydroxide.

Aryl hydrazines of formula (VIII) are commercially available or may be made according to methods known by those skilled in the art.

Compounds of formula (I) may be prepared in the form of mixtures of enantiomers when mixtures of enantiomers are used as intermediates in the synthesis. The 2R isomer of the compounds of the invention may be isolated from the mixtures by conventional techniques, for example HPLC on a chiral column.

Alternatively, separation of enantiomers may be performed earlier in the synthesis, for example, individual enantiomers of compounds of intermediates (III), (IV) or (VI) may be employed which may obviate the need to perform a separation of enantiomers as a final stage in the synthesis. The later process is, in theory, more efficient and is therefore preferred.

Certain compounds of formula (II), for example 5-amino-1-(2,4-difluorophenyl)-1H-pyrazole-4-carboxylic acid (compound of formula (II), wherein R¹=R²=F), are also new and form an aspect of the present invention.

The compounds of formula (III) are new and form an aspect of the present invention.

Certain compounds of formula (IV) and (V) are also new, both as mixtures of isomers and single enantiomers or diastereomers and form a further aspect of the present invention.

Compositions comprising a compound of the invention also constitute an aspect of the invention.

In addition, processes for preparing formulations including one or more compounds of the invention form an aspect of this invention.

Compounds of formula (I) or derivatives thereof, which are not physiologically acceptable, may be useful as intermediates in the preparation of other compounds of formula (I) or physiologically functional derivatives thereof.

Compounds of the invention may be expected to demonstrate good anti-inflammatory properties, with predictable pharmacokinetic and pharmacodynamic behaviour. They also may be expected to have an attractive side-effect profile, demonstrated, for example, by increased selectivity for the glucocorticoid receptor over the progesterone receptor and/or increased selectivity for glucocorticoid receptor mediated transrepression over transactivation and are expected to be compatible with a convenient regime of treatment in human patients.

The invention will now be illustrated by way of the following non-limiting examples.

### EXAMPLES

### Abbreviations

| | |
|---|---|
| EtOAc | Ethyl acetate |
| IPA | Isopropyl alcohol |
| EtOH | Ethanol |
| DIPEA | Diisopropylethylamine |
| DCM | Dichloromethane |
| NaOH | Sodium hydroxide |
| SPE | Solid phase extraction |
| HATU | O-(7-Azobenzotriazol-1-yl)-1,1,3,3-tetramethyl uronium |
| | hexafluorophosphate |
| DMF | N,N-dimethylformamide |
| MeOH | Methanol |
| MeCN | Acetonitrile |
| TFA | Trifluoroacetic acid |
| NH₃ | Ammonia |
| DMSO | Dimethylsulphoxide |
| RT | Room Temperature |

LCMS System

The LCMS system used was as follows:
Column: 3.3 cm×4.6 mm ID, 3 μm ABZ+PLUS from Supelco
Flow Rate: 3 ml/min
Injection Volume: 5 μl
Temp: Room Temperature
UV Detection Range: 215 to 330 nm
   Solvents: A: 0.1% Formic Acid+10 mMolar Ammonium Acetate.
B: 95% Acetonitrile+0.05% Formic Acid

| | Time | A % | B % |
|---|---|---|---|
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

### Mass-Directed Autopreparation

### System 1

Purifications were carried out using a Micromass ZQ platform. The column was a 100 mm×20 mm Supelco LCABZ++ with stationary phase particle size of 5 μm.

| | |
|---|---|
| Solvents: | A: water + 0.1% formic acid |
| | B: MeCN:water 95:5 + 0.05% formic acid |
| Gradient | 50-90% B over 10 minutes |
| Flow rate | 20 mL/min |

### System 2

Purifications were carried out using an Agilent 1100 series platform. The column was a 212 mm×100 mm Zorbax Eclipse XDB-C18 prep HT with stationary phase particle size of 5 μm.

| | |
|---|---|
| Solvents: | A: water + 0.1% TFA |
| | B: MeCN + 0.1% TFA |
| Gradient | 90% A for 1 minute changing to 95% B over 9 minutes |
| Flow rate | 20 mL/min |

### Circular Dichroism

Circular dichroism was carried out on an Applied Photophysics Chirascan spectrophotometer at room temperature, using acetonitrile as solvent, over the range 200-350 nm

### Intermediates 1 and 2: 4-(2,3-Dihydro-1-benzofuran-7-yl)-1,1,1-trifluoro-4-methyl-2-({[(1R)-1-phenylethyl]amino}methyl)-2-pentanol

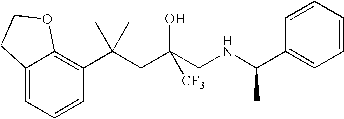

To an ice-cooled solution of (R)-(+)-1-phenylethylamine (4.43 mL, 34.9 mmol) in anhydrous EtOH (3 mL) was added, dropwise, racemic 7-{1,1-dimethyl-2-[2-(trifluoromethyl)-2-oxiranyl]ethyl}-2,3-dihydro-1-benzofuran (WO04/063163, 1 g, 3.49 mmol). The reaction mixture was then heated at 50° C. overnight, cooled to room temperature and evaporated in vacuo. The residue was applied to a 50 g silica SPE cartridge and eluted with 0.5% NH₃ in toluene. The appropriate fractions were combined and evaporated in vacuo to give a colourless oil (1.486 g). This oil was subjected to mass-directed autopreparation (system 1) to give Intermediate 1 (2S isomer, 314 mg), Intermediate 2 (2R isomer, 334 mg) plus a mixed fraction (480 mg). The mixed fraction was re-subjected to mass-directed autopreparation to give further Intermediate 1 (90 mg), Intermediate 2 (125 mg) plus a mixed fraction (160 mg). The fractions containing Intermediate 1 were combined with each other as were the fractions containing Intermediate 2.

### Intermediate 1 (2S Isomer):

Single crystal X-ray structure on a triclinic crystal obtained by slow evaporation from isopropanol established the 2S configuration.

LCMS: t_{RET}=2.86 min; MH⁺=408, melting point 65 to 68° C.

### Intermediate 2 (2R Isomer):

LCMS: t_{RET}=2.94 min; MH⁺=408

### Intermediate 3: (2R)-2-(Aminomethyl)-4-(2,3-dihydro-1-benzofuran-7-yl)-1,1,1-trifluoro-4-methyl-2-pentanol

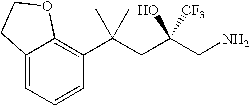

(2R)-4-(2,3-Dihydro-1-benzofuran-7-yl)-1,1,1-trifluoro-4-methyl-2-({[(1R)-1-phenylethyl]amino}methyl)-2-pentanol (Intermediate 2) (480 mg, 1.178 mmol) was dissolved in EtOH (13 mL) and hydrogenated over 10% palladium on charcoal (119 mg) at 50 psi and room temperature for 5 hours. Catalyst was removed by filtration through a microfibre filter pad and Celite. The Celite was washed several times with EtOH. The filtrate was evaporated in vacuo to give the title compound as a pale grey solid (330 mg) which was used without further purification.

LCMS: t_{RET}=2.43 min; MH⁺=304

### Intermediates 4 and 5: Phenylmethyl[4-[5-fluoro-2-(methyloxy)phenyl]-2-hydroxy-4-methyl-2-(trifluoromethyl)pentyl]carbamate

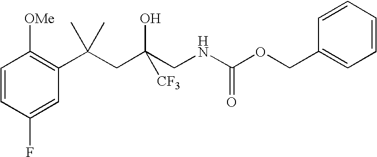

Racemic 2-(aminomethyl)-1,1,1-trifluoro-4-[5-fluoro-2-(methyloxy)phenyl]-4-methyl-2-pentanol (WO03/082827, 894 mg, 2.89 mmol) was dissolved in DCM (15 mL). N-(benzyloxycarbonyloxy)succinimide (1.08 g, 4.34 mmol) was added and the mixture was stirred for 5 min. Triethylamine (804 μL, 5.78 mmol) was added and stirring was continued for 3.5 hours after which time the reaction mixture was washed with saturated sodium bicarbonate solution and evaporated in vacuo. Purification of the residue by Flashmaster 11 using a 10 g silica cartridge and a solvent gradient of cyclohexane:EtOAc 100:0 to 0:100 over a period of 1 hour gave the racemic product as an oil (1.12 g). This oil was applied to a 2 inch×20 cm Chiralpak AD column eluted with heptane: IPA 97.5:2.5 with a flow rate of 75 mL/min to give Intermediate 4 (2R isomer, 443 mg) after ca. 41 min and Intermediate 5 (2S isomer, 441 mg) after ca. 60 min.

Intermediate 4 (2R isomer): Analytical chiral HPLC (25×0.46 cm Chiralpak AD column,

heptane: IPA 97.5:2.5 eluting at 1 mL/min): 7.9 min

LCMS: t_{RET}=3.75 min; MH⁺=444.

Intermediate 5 (2S isomer): Analytical chiral HPLC (25×0.46 cm Chiralpak AD column,

heptane: IPA 97.5:2.5 eluting at 1 mL/min): 9.8 min

LCMS: t_{RET}=3.75 min; MH⁺=444.

### Intermediates 6 and 7: 1,1,1-Trifluoro-4-[5-fluoro-2-(methyloxy)phenyl]-4-methyl-2-({[(1R)-1-phenylethyl]amino}methyl)-2-pentanol

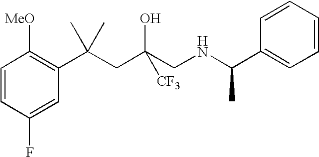

To a stirred solution of racemic 2-{2-[5-fluoro-2-(methyloxy)phenyl]-2-methylpropyl}-2-(trifluoromethyl)oxirane (WO04/063163, 600 mg, 2.05 mmol) in anhydrous EtOH (3 mL) was added (R)-(+)-1-phenylethylamine (1.31 mL, 10.3 mmol). The reaction mixture was then stirred and heated at 50° C. under nitrogen for 5 days, cooled to room temperature and evaporated in vacuo. The residue was applied to a 70 g silica SPE cartridge and eluted with 0.5% NH₃ in toluene. The appropriate fractions were combined and evaporated in vacuo to give a colourless oil (991 mg). 710 mg of this oil was separated by chiral HPC on a 2 inch×15 cm Chiralpak AD column eluted with 25% acetonitrile/ammonium phosphate (pH 4.9) with a flow rate of 70 mL/min to give Intermediate 6 (2S isomer, 230 mg) after 17.5 min and Intermediate 7 (2R isomer, 200 mg) after 24.8 min.

Intermediate 6 (2S isomer):

Single crystal X-ray structure on an orthorhombic crystal obtained by slow evaporation from EtOAc established the 2S configuration.

LCMS: t_{RET}=2.81 min; MH⁺=414

Intermediate 7 (2R isomer):

LCMS: t_{RET}=2.91 min; MH⁺=414

### Intermediate 8: (2R)-2-(Aminomethyl)-1,1,1-trifluoro-4-[5-fluoro-2-(methyloxy)phenyl]-4-methyl-2-pentanol

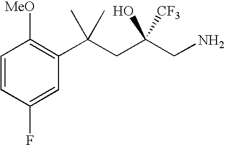

### Method A: via (2R)-phenylmethyl[4-[5-fluoro-2-(methyloxy)phenyl]-2-hydroxy-4-methyl-2-(trifluoromethyl)pentyl]carbamate (Intermediate 4)

(2R)-Phenylmethyl[4-[5-fluoro-2-(methyloxy)phenyl]-2-hydroxy-4-methyl-2-(trifluoromethyl)pentyl]carbamate (Intermediate 4) (343 mg, 0.774 mmol) was dissolved in EtOH (25 mL) and hydrogenated over 10% palladium on charcoal (34 mg) at room temperature and 3 bar for 4 hours. The catalyst was removed by filtration through Celite and the filtrate was evaporated in vacuo to give the title compound as a grey solid (213 mg) which was used without further purification.

LCMS: t_{RET}=2.38 min; MH⁺=310

Circular Dichroism (Cell Length: 0.5 cm; Concentration: 230 μM)

221.0 nm (de =−1.25) and 280.4 nm (de =−0.76)

### Method B: via (2R)-1,1,1-trifluoro-4-[5-fluoro-2-(methyloxy)phenyl]-4-methyl-2-({[(1R)-1-phenylethyl]amino}methyl)-2-pentanol (Intermediate 7)

(2R)-1,1,1-Trifluoro-4-[5-fluoro-2-(methyloxy)phenyl]-4-methyl-2-({[(1R)-1-phenylethyl]amino}methyl)-2-pentanol (Intermediate 7) (200 mg, 0.48 mmol) was dissolved in EtOH (8 mL) and hydrogenated over 10% palladium on charcoal (100 mg) at 53 psi and room temperature for 16 hours. Catalyst was removed by filtration through Celite. The Celite was washed several times with EtOH. The filtrate was evaporated in vacuo to give the title compound as a pale yellow oil (158 mg) which was used without further purification.

LCMS: t_{RET}=2.38 min; MH⁺=310

Circular Dichroism (Cell Length: 0.5 cm; Concentration: 230 μM)

222.0 nm (de=−0.96) and 280.8 nm (de=−0.69)

### Intermediate 9: Ethyl 5-Amino-1-(2,4-difluorophenyl)-1H-pyrazole-4-carboxylate

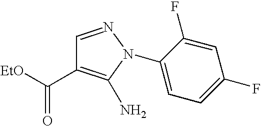

2,4-Difluorophenylhydrazine hydrochloride (9.24 g, 51.17 mmol) and ethyl (ethoxymethylene)cyanoacetate (10.35 g, 61.41 mmol) were stirred together with EtOH (200 mL). DIPEA (7.94 g, 61.41 mmol) was added and the mixture was stirred and refluxed overnight, cooled and evaporated in vacuo. The residue was partitioned between DCM and water. The organic phase was separated, washed with brine, dried over sodium sulphate and evaporated to give a dark brown solid (15.17 g). This material was combined with a further 1.29 g from an additional preparation of this substance. Purification on a silica column eluting with a solvent gradient of 10-20% EtOAc in hexane gave the title compound as a yellow solid (8.46 g).

LCMS: t_{RET}=2.70 min; MH⁺=268

### Intermediate 10: 5-Amino-1-(2,4-difluorophenyl)-1H-pyrazole-4-carboxylic acid

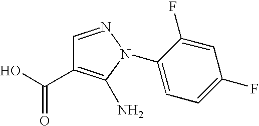

A mixture of ethyl 5-amino-1-(2,4-difluorophenyl)-1H-pyrazole-4-carboxylate (Intermediate 9) (6.8 g, 25.5 mmol), 1M NaOH solution (100 mL) and EtOH (20 mL) was heated at 70° C. for 2.5 hours, cooled and the pH adjusted to 7 with 2M hydrochloric acid (50 mL). The precipitated solid was filtered off, washed with water and dried at 40° C. in vacuo over the weekend to give the title compound (5.1 g) which was used without further purification.

LCMS: t_{RET}=2.20 min; MH⁺=240

### Intermediate 11: 1,1,1-Trifluoro-4-[5-fluoro-2-(methyloxy)phenyl]-4-methyl-2-{[(phenylmethyl)amino]methyl}-2-pentanol

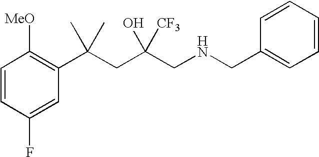

Benzylamine (38.6 mL, 0.353 mol) was added in one portion to a stirred solution of racemic 2-{2-[5-fluoro-2-(methyloxy)phenyl]-2-methylpropyl}-2-(trifluoromethyl)oxirane (which may be prepared according to WO 04/063163, 50 g, 0.171 mol) in EtOH (500 mL) at 20° C. and the resulting mixture heated at 80° C. overnight. The solvent was removed under reduced pressure and the resulting oil was purified by silica gel column chromatography eluting with 4% EtOAc in cyclohexane to give the title compound as a white solid (65.1 g). LCMS: t_{RET}=2.83 min; MH⁺=400

### Intermediate 12: 2-(Aminomethyl)-1,1,1-trifluoro-4-[5-fluoro-2-(methyloxy)phenyl]-4-methyl-2-pentanol

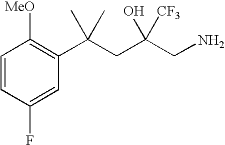

1,1,1-Trifluoro-4-[5-fluoro-2-(methyloxy)phenyl]-4-methyl-2-{[(phenylmethyl)amino]methyl}-2-pentanol (Intermediate 17, 63 g, 0.158 mol) was added to a stirred solution of 10% Palladium on charcoal (12.6 g, 50% wet) in EtOH (1.07 L) at 20° C. in a nitrogen purged vessel. The mixture was hydrogenated at 20° C. and atmospheric pressure until there was no further hydrogen uptake. The suspension was then filtered through Celite and glass fibre filter paper to remove the catalyst, and the cake washed with EtOH (120 mL). The combined filtrate and washings were evaporated under reduced pressure to give the title compound as a light grey solid (47.5 g).

LCMS: t_{RET}=2.37 min; MH⁺=310

### Intermediate 13: 5-Amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide

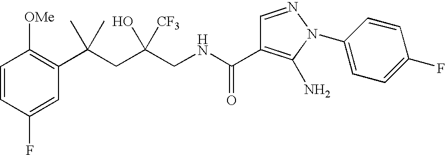

HATU (28.1 g, 73.9 mmol) was added to a stirred mixture of 5-amino-1-(4-fluorophenyl)-1H-pyrazole-4-carboxylic acid (16.5 g, 74.6 mmol) and DIPEA (38 mL, 218 mmol) in DMF (230 mL) at 20° C. The mixture was stirred at 20° C. for 20 min when 2-(aminomethyl)-1,1,1-trifluoro-4-[5-fluoro-2-(methyloxy)phenyl]-4-methyl-2-pentanol (Intermediate 12, 23 g, 74.4 mmol) was added. The resulting solution was stirred overnight and then poured into water (700 mL) and extracted with diisopropyl ether (2×350 mL). The organic phase was separated, washed successively with water (350 mL) and brine (350 mL), and then dried over anhydrous sodium sulphate and the solvent evaporated under reduced pressure. The residue was purified by silica gel column chromatography eluting with 20% EtOAc in cyclohexane to afford the title compound as a white solid (33.2 g)

LCMS: t_{RET}=3.57 min; MH⁺=513

### Example 1

### (2R)-5-Amino-N-[4-(2,3-dihydro-1-benzofuran-7-yl)-2-hydroxy-4-methyl-2-(trifluoromethyl)pentyl]-1-phenyl-1H-pyrazole-4-carboxamide

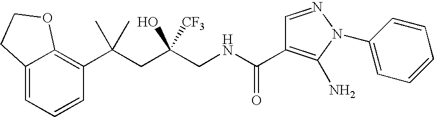

5-Amino-1-phenyl-1H-pyrazole-4-carboxylic acid (18 mg, 0.087 mmol) was dissolved in anhydrous DMF (1.5 mL) and DIPEA (29 μL, 0.168 mmol) was added followed by HATU (33 mg, 0.087 mmol). The reaction was left to stir at room temperature under nitrogen for 10 minutes then a solution of (2R)-2-(aminomethyl)-4-(2,3-dihydro-1-benzofuran-7-yl)-1,1,1-trifluoro-4-methyl-2-pentanol (Intermediate 3) (20.4 mg, 0.067 mmol) in anhydrous DMF (0.5 mL) was added. The reaction mixture was stirred for 18 hours then partitioned between 1:1 brine/water and EtOAc. The organic layer was separated, passed through a hydrophobic frit and evaporated in vacuo to give a yellow residue. Mass-directed autopreparation (system 1) gave the title compound (27 mg).

LCMS: t_{RET}=3.70 min; MH⁺=489

### Example 2

### (2R)-5-Amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide

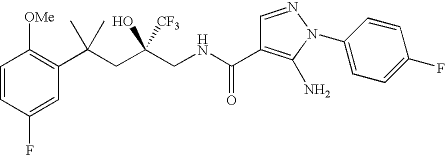

### Method A

To a solution of HATU (27.4 mg, 0.072 mmol) in DMF (200 μL) was added DIPEA (21 μL, 0.12 mmol) and the resulting solution was added to 5-amino-1-(4-fluorophenyl)-1H-pyrazole-4-carboxylic acid (15.9 mg, 0.072 mmol) and the mixture was shaken for 10 mins. A solution of (2R)-2-(aminomethyl)-1,1,1-trifluoro-4-[5-fluoro-2-(methyloxy)phenyl]-4-methyl-2-pentanol (Intermediate 8) (18.6 mg, 0.06 mmol) in DMF (200 μL) was added and the mixture was shaken for 10 mins then left to stand for 18 hours. DMF was removed in vacuo and the residue was dissolved in chloroform (300 μL) and applied to a 500 mg aminopropyl SPE cartridge. Elution with chloroform (2 mL) followed by evaporation of the chloroform fractions gave the title compound (32.8 mg).

LCMS: t_{RET}=3.66 min; MH⁺=513

### Method B

5-Amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide (Intermediate 13, 1.3 g) was separated into it's enantiomers by preparative chiral HPLC on a 21×5 cm Chiralcel OD 20 micron column. The racemate was dissolved in tert-butyl methyl ether (14 mL) for injection onto the column which was eluted with 75:25 n-heptane:EtOH at a flow rate of 75 mL/min for 6 min and then at 150 mL/min for 3 min. The second eluting 2R enantiomer, was collected in a fraction eluting between 6.6 min and 8.2 min which was evaporated to afford the title compound as a white solid (585 mg).

LCMS: t_{RET}=3.57 min; MH⁺=513. Analytical chiral HPLC using a Chiralcel OD analytical column (250×4.6 mm) eluting with 70:30 n-heptane:EtOH at 0.8 mL/min and a column temperature of 25° C. indicated chiral purity of 99.3%.

### Example 3

### (2R)-5-Amino-N-[4-[5-fluoro-2-(methyloxy)phenyl]-2-hydroxy-4-methyl-2-(trifluoromethyl)pentyl]-1-(2-fluorophenyl)-1H-pyrazole-4-carboxamide

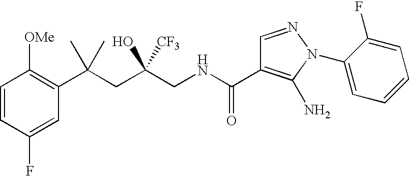

Prepared similarly to Example 2 from (2R)-2-(aminomethyl)-1,1,1-trifluoro-4-[5-fluoro-2-(methyloxy)phenyl]-4-methyl-2-pentanol (Intermediate 8) and 5-amino-1-(2-fluorophenyl)-1H-pyrazole-4-carboxylic acid.

LCMS: t_{RET}=3.61 min; MH⁺=513

### Example 4

### (2R)-5-Amino-1-(2,4-difluorophenyl)-N-[4-[5-fluoro-2-(methyloxy)phenyl]-2-hydroxy-4-methyl-2-(trifluoromethyl)pentyl]-1H-pyrazole-4-carboxamide

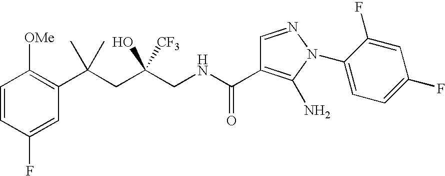

Prepared similarly to Example 2 from (2R)-2-(aminomethyl)-1,1,1-trifluoro-4-[5-fluoro-2-(methyloxy)phenyl]-4-methyl-2-pentanol (Intermediate 8) and 5-amino-1-(2,4-difluorophenyl)-1H-pyrazole-4-carboxylic acid (Intermediate 10) except that additional purification by mass-directed autopreparation (system 2) was employed.

LCMS: t_{RET}=3.64 min; MH⁺=531

### Example 5

### (2R)-5-Amino-1-(4-chlorophenyl)-N-[4-[5-fluoro-2-(methyloxy)phenyl]-2-hydroxy-4-methyl-2-(trifluoromethyl)pentyl]-1H-pyrazole-4-carboxamide

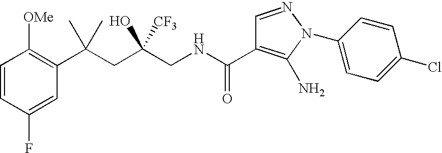

Prepared similarly to Example 2 from (2R)-2-(aminomethyl)-1,1,1-trifluoro-4-[5-fluoro-2-(methyloxy)phenyl]-4-methyl-2-pentanol (Intermediate 8) and 5-amino-1-(4-chlorophenyl)-1H-pyrazole-4-carboxylic acid.

LCMS: t_{RET}=3.79 min; MH⁺=529/531

### Example 6

### (2R)-5-Amino-N-[4-[5-fluoro-2-(methyloxy)phenyl]-2-hydroxy-4-methyl-2-(trifluoromethyl)pentyl]-1-phenyl-1H-pyrazole-4-carboxamide

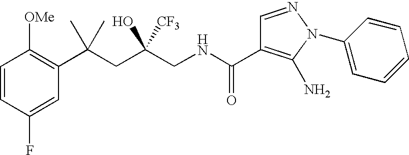

Prepared similarly to Example 2 from (2R)-2-(aminomethyl)-1,1,1-trifluoro-4-[5-fluoro-2-(methyloxy)phenyl]-4-methyl-2-pentanol (Intermediate 8) and 5-amino-1-phenyl-1H-pyrazole-4-carboxylic acid.

LCMS: t_{RET}=3.65 min; MH⁺=495

### Example 7

### (2R)-5-Amino-N-[4-(5-fluoro-2-hydroxyphenyl)-2-hydroxy-4-methyl-2-(trifluoromethyl)pentyl]-1-(4-fluorophenyl)-1H-pyrazole-4-carboxamide

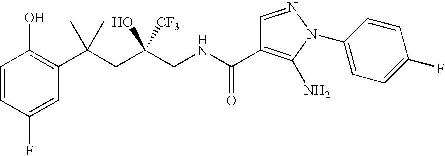

(2R)-5-Amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide (Example 2) (20.4 mg) was dissolved in DCM (200 μL). A solution of boron tribromide (1M in DCM, 500 μL) was added and the mixture was left to stand for 18 hours before being cooled (ice) and quenched with methanol (0.5 mL). The mixture was evaporated and the residue was purified by mass-directed autopreparation (system 2) to give the title compound (12.4 mg).

LCMS: t_{RET}=3.50 min; MH⁺=499

### Example 8

### (2R)-5-Amino-N-[4-(5-fluoro-2-hydroxyphenyl)-2-hydroxy-4-methyl-2-(trifluoromethyl)pentyl]-1-(2-fluorophenyl)-1H-pyrazole-4-carboxamide

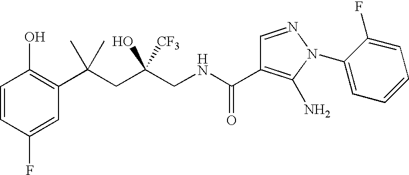

Similarly prepared to Example 7 from (2R)-5-amino-N-[4-[5-fluoro-2-(methyloxy)phenyl]-2-hydroxy-4-methyl-2-(trifluoromethyl)pentyl]-1-(2-fluorophenyl)-1H-pyrazole-4-carboxamide (Example 3).

LCMS: t_{RET}=3.44 min; MH⁺=499

### Example 9

### (2R)-5-Amino-1-(2,4-difluorophenyl)-N-[4-(5-fluoro-2-hydroxyphenyl)-2-hydroxy-4-methyl-2-(trifluoromethyl)pentyl]-1H-pyrazole-4-carboxamide

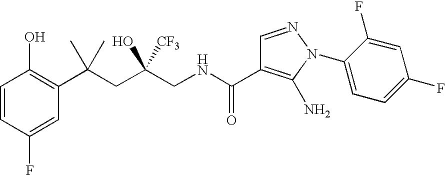

Similarly prepared to Example 7 from (2R)-5-amino-1-(2,4-difluorophenyl)-N-[4-[5-fluoro-2-(methyloxy)phenyl]-2-hydroxy-4-methyl-2-(trifluoromethyl)pentyl]-1H-pyrazole-4-carboxamide (Example 4).

LCMS: t_{RET}=3.46 min; MH⁺=517

### Example 10

### (2R)-5-Amino-1-(4-chlorophenyl)-N-[4-(5-fluoro-2-hydroxyphenyl)-2-hydroxy-4-methyl-2-(trifluoromethyl)pentyl]-1H-pyrazole-4-carboxamide

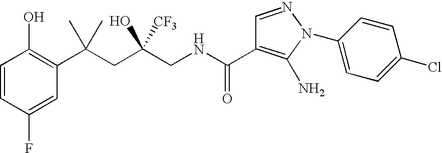

Similarly prepared to Example 7 from (2R)-5-amino-1-(4-chlorophenyl)-N-[4-[5-fluoro-2-(methyloxy)phenyl]-2-hydroxy-4-methyl-2-(trifluoromethyl)pentyl]-1H-pyrazole-4-carboxamide (Example 5).

LCMS: t_{RET}=3.64 min; MH⁺=515/517

### Example 11

### (2R)-5-Amino-N-[4-(5-fluoro-2-hydroxyphenyl)-2-hydroxy-4-methyl-2-(trifluoromethyl)pentyl]-1-phenyl-1H-pyrazole-4-carboxamide

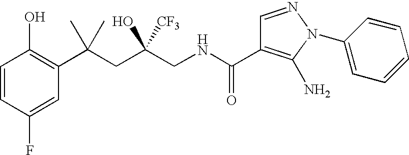

Similarly prepared to Example 7 from (2R)-5-amino-N-[4-[5-fluoro-2-(methyloxy)phenyl]-2-hydroxy-4-methyl-2-(trifluoromethyl)pentyl]-1-phenyl-1H-pyrazole-4-carboxamide (Example 6).

LCMS: t_{RET}=3.48 min; MH⁺=481

### Example 12

### (2R)-5-Amino-N-[4-(2,3-dihydro-1-benzofuran-7-yl)-2-hydroxy-4-methyl-2-(trifluoromethyl)pentyl]-1-(2-fluorophenyl)-1H-pyrazole-4-carboxamide

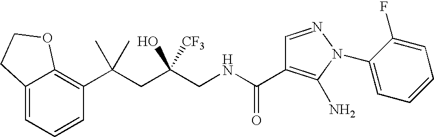

Prepared similarly to Example 2 from (2R)-2-(aminomethyl)-4-(2,3-dihydro-1-benzofuran-7-yl)-1,1,1-trifluoro-4-methyl-2-pentanol (Intermediate 3) and 5-amino-1-(2-fluorophenyl)-1H-pyrazole-4-carboxylic acid.

LCMS: t_{RET}=3.51 min; MH⁺=507

### Example 13

### (2R)-5-Amino-N-[4-(2,3-dihydro-1-benzofuran-7-yl)-2-hydroxy-4-methyl-2-(trifluoromethyl)pentyl]-1-(4-fluorophenyl)-1H-pyrazole-4-carboxamide

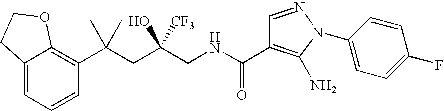

Prepared similarly to Example 2 from (2R)-2-(aminomethyl)-4-(2,3-dihydro-1-benzofuran-7-yl)-1,1,1-trifluoro-4-methyl-2-pentanol (Intermediate 3) and 5-amino-1-(4-fluorophenyl)-1H-pyrazole-4-carboxylic acid.

LCMS: t_{RET}=3.57 min; MH⁺=507

### Example 14

### (2R)-5-Amino-1-(2,4-difluorophenyl)-N-[4-(2,3-dihydro-1-benzofuran-7-yl)-2-hydroxy-4-methyl-2-(trifluoromethyl)pentyl]-1H-pyrazole-4-carboxamide

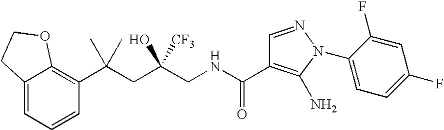

Prepared similarly to Example 2 from (2R)-2-(aminomethyl)-4-(2,3-dihydro-1-benzofuran-7-yl)-1,1,1-trifluoro-4-methyl-2-pentanol (Intermediate 3) and (2R)-5-amino-1-(4-chlorophenyl)-N-[4-(5-fluoro-2-hydroxyphenyl)-2-hydroxy-4-methyl-2-(trifluoromethyl)pentyl]-1H-pyrazole-4-carboxamide (Intermediate 10).

LCMS: t_{RET}=3.54 min; MH⁺=525

### Example 15

### (2R)-5-Amino-1-(4-chlorophenyl)-N-[4-(2,3-dihydro-1-benzofuran-7-yl)-2-hydroxy-4-methyl-2-(trifluoromethyl)Pentyl]-1H-pyrazole-4-carboxamide

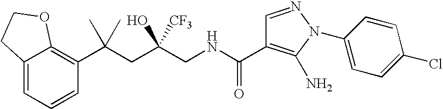

Prepared similarly to Example 2 from (2R)-2-(aminomethyl)-4-(2,3-dihydro-1-benzofuran-7-yl)-1,1,1-trifluoro-4-methyl-2-pentanol (Intermediate 3) and 5-amino-1-(4-chlorophenyl)-1H-pyrazole-4-carboxylic acid except that additional purification by mass-directed autopreparation (system 2) was employed.

LCMS: t_{RET}=3.70 min; MH⁺=523/525

### BIOLOGICAL EXPERIMENTAL

### Glucocorticoid Receptor Binding Assay

The ability of compounds to bind to the glucocorticoid receptor was determined by assessing their ability to compete with an Alexa 555 fluorescently-labelled dexamethasone derivative. Compounds were solvated and diluted in DMSO, and transferred directly into assay plates. Fluorescent dexamethasone and a partially purified full length glucocorticoid receptor were added to the plates, together with buffer components to stabilise the GR protein and incubated at room temperature for 2 hours in the dark. Binding of each compound was assessed by analysing the displacement of fluorescent ligand by measuring the decrease in fluorescence polarisation signal from the mixture.

Examples 1-15 have glucocorticoid binding with a pIC₅₀>7.5 in this assay.

### Glucocorticoid Mediated Transrepression of NFkB Activity

Human A549 lung epithelial cells were engineered to contain a secreted placental alkaline phosphatase gene under the control of the distal region of the NFkB dependent ELAM promoter as previously described in Ray, K. P., Farrow, S., Daly, M., Talabot, F. and Searle, N. “Induction of the E-selectin promoter by interleukin 1 and tumour necrosis factor alpha, and inhibition by glucocorticoids” Biochemical Journal (1997) 328: 707-15.

Compounds were solvated and diluted in DMSO, and transferred directly into assay plates such that the final concentration of DMSO was 0.7%. Following the addition of cells (40K per well), plates were incubated for 1 hour prior to the addition of 3 ng/ml human recombinant TNFα. Following continued incubation for 16 hours, alkaline phosphatase activity was determined by measuring the change in optical density at 405 nM with time following the addition of 0.7 volumes of assay buffer (1 mg/ml p-nitrophenylphosphate dissolved in 1M diethanolamine, 0.28M NaCl, 0.5 mM MgCl₂). Dose response curves were constructed from which EC₅₀ values were estimated.

Examples 1-15 show pEC₅₀>8.5 in this assay

### Assay for Progesterone Receptor Activity

A T225 flask of CV-1 cells at a density of 80% confluency was washed with PBS, detached from the flask using 0.25% trypsin and counted using a Sysmex KX-21N. Cells were diluted in DMEM containing 10% Hyclone, 2 mM L-Glutamate and 1% Pen/Strep at 140 cells/μl and transduced with 10% PRb-BacMam and 10% MMTV-BacMam. 70 ml of suspension cells were dispensed to each well of white Nunc 384-well plates, containing compounds at the required concentration. After 24 hours 10 μl of Steady Glo were added to each well of the plates. Plates were incubated in the dark for 10 minutes before reading them on a Viewlux reader. Dose response curves were constructed from which pEC₅₀ values were estimated.

Examples 1-11 show pEC₅₀<6 in this assay.

In describing those examples which are preferred or more preferred according to their activity in the assays above, it will be appreciated that at least one isomer, for example, an enantiomer in a mixture of isomers (such as a racemate) has the described activity. The other enantiomer may have similar activity, less activity, no activity or may have some antagonist activity in the case of a functional assay.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

The patents and patent applications described in this application are herein incorporated by reference.

What is claimed is:

1. A compound of formula (I): 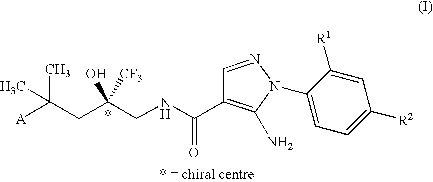
wherein
A represents 2,3-dihydro-1-benzofuran-7-yl, 5-fluoro-2-methoxy-phenyl or 5-fluoro-2-hydroxy-phenyl; and
R¹ and R² each independently represent hydrogen, fluorine or chlorine; or
a physiologically functional derivative thereof.

2. A compound according to claim 1 which is substantially free of the corresponding 2S isomer.

3. A compound according to claim 1 containing less than 5% by weight of the corresponding 2S isomer.

4. A compound according to claim 1 containing less than 2% by weight of the
corresponding 2S isomer.

5. A compound according to claim 1 containing less than 1% by weight of
the corresponding 2S isomer.

6. (canceled)

7. A compound which is selected from the group consisting of:
(2R)-5-amino-N-[4-(2,3-dihydro-1-benzofuran-7-yl)-2-hydroxy-4-methyl-2-(trifluoromethyl)pentyl]-1-phenyl-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-(4-fluorophenyl)-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-(2-fluorophenyl)-1H-pyrazole-4-carboxamide;
(2R)-5-amino-1-(2,4-difluorophenyl)-N-[4-[5-fluoro-2-(methyloxy)phenyl]-2-hydroxy-4-methyl-2-(trifluoromethyl)pentyl]-1H-pyrazole-4-carboxamide;
(2R)-5-amino-1-(4-chlorophenyl)-N-[4-[5-fluoro-2-(methyloxy)phenyl]-2-hydroxy-4-methyl-2-(trifluoromethyl)pentyl]-1H-pyrazole-4-carboxamide;
(2R)-5-amino-N-[4-[5-fluoro-2-(methyloxy)phenyl]-2-hydroxy-4-methyl-2-(trifluoromethyl)pentyl]-1-phenyl-1H-pyrazole-4-carboxamide;
(2R)-5-amino-N-[4-(5-fluoro-2-hydroxyphenyl)-2-hydroxy-4-methyl-2-(trifluoromethyl)pentyl]-1-(4-fluorophenyl)-1H-pyrazole-4-carboxamide;
(2R)-5-amino-N-[4-(5-fluoro-2-hydroxyphenyl)-2-hydroxy-4-methyl-2-(trifluoromethyl)pentyl]-1-(2-fluorophenyl)-1H-pyrazole-4-carboxamide;
(2R)-5-amino-1-(2,4-difluorophenyl)-N-[4-(5-fluoro-2-hydroxyphenyl)-2-hydroxy-4-methyl-2-(trifluoromethyl)pentyl]-1H-pyrazole-4-carboxamide;
(2R)-5-amino-1-(4-chlorophenyl)-N-[4-(5-fluoro-2-hydroxyphenyl)-2-hydroxy-4-methyl-2-(trifluoromethyl)pentyl]-1H-pyrazole-4-carboxamide;
(2R)-5-amino-N-[4-(5-fluoro-2-hydroxyphenyl)-2-hydroxy-4-methyl-2-(trifluoromethyl)pentyl]-1-phenyl-1H-pyrazole-4-carboxamide;
(2R)-5-amino-N-[4-(2,3-dihydro-1-benzofuran-7-yl)-2-hydroxy-4-methyl-2-(trifluoromethyl)pentyl]-1-(2-fluorophenyl)-1H-pyrazole-4-carboxamide;
(2R)-5-amino-N-[4-(2,3-dihydro-1-benzofuran-7-yl)-2-hydroxy-4-methyl-2-(trifluoromethyl)pentyl]-1-(4-fluorophenyl)-1H-pyrazole-4-carboxamide;
(2R)-5-amino-1-(2,4-difluorophenyl)-N-[4-(2,3-dihydro-1-benzofuran-7-yl)-2-hydroxy-4-methyl-2-(trifluoromethyl)pentyl]-1H-pyrazole-4-carboxamide;
(2R)-5-amino-1-(4-chlorophenyl)-N-[4-(2,3-dihydro-1-benzofuran-7-yl)-2-hydroxy-4-methyl-2-(trifluoromethyl)pentyl]-1H-pyrazole-4-carboxamide; and
a physiologically functional derivative thereof.

8-12. (canceled)

13. A method for the treatment of a human subject with an inflammatory condition, which method comprises administering to said human or animal subject an effective amount of a compound as claimed in claim 1, or a physiologically functional derivative thereof.

14. A method for the treatment of a human subject with skin disease, which method comprises administering to said human or animal subject an effective amount of a compound as claimed in claim 1, or a physiologically functional derivative thereof.

15. A pharmaceutical composition comprising a compound as claimed in claim 1, or a physiologically functional derivative thereof, in admixture with one or more physiologically acceptable diluents or carriers.

16. A pharmaceutical aerosol formulation comprising a compound as defined in claim 1, or a physiologically functional derivative thereof, and a fluorocarbon or hydrogen-containing chlorofluorocarbon as propellant, optionally in combination with a surfactant and/or a cosolvent.

17. A pharmaceutical aerosol formulation as claimed in claim 16 wherein the propellant is selected from the group consisting of 1,1,1,2-tetrafluoroethane, 1,1,1,2,3,3,3-heptafluoro-n-propane and mixtures thereof.

18. A combination comprising a compound as claimed in claim 1, or a physiologically functional derivative thereof, together with one or more other therapeutically active agents.

19. A combination according to claim 18 in which said therapeutically active agent is a β₂-adrenoreceptor agonist.

20. A combination according to claim 18 in which said therapeutically active agent is PDE4 inhibitor.

21. A combination according to claim 18 in which said therapeutically active agent is an antihistamine.

22. A process for the preparation of a compound of as claimed in claim 1, or a physiologically functional derivative thereof, comprising coupling of a carboxylic acid of formula (II): 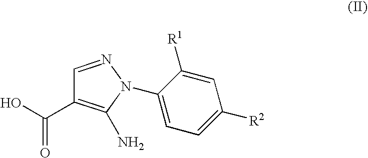
or an activated derivative thereof
wherein the groups R¹ and R² are as defined for compounds of formula (I),
with an amine of formula (III): 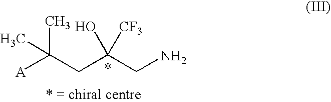
or a protected derivative thereof
wherein the group A is as defined for compounds of formula (I).

23. A compound of formula (II) 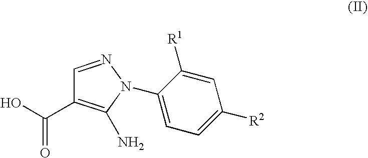
wherein R¹ and R² each independently represent hydrogen, fluorine or chlorine.

24. A compound of formula (III) 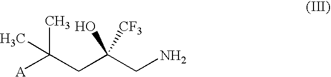
wherein A represents 2,3-dihydro-1-benzofuran-7-yl, 5-fluoro-2-methoxy-phenyl or 5-fluoro-2-hydroxy-phenyl.

25. A compound of formula (IV) 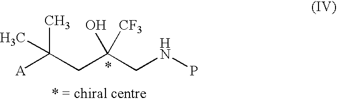
wherein A represents 2,3-dihydro-1-benzofuran-7-yl, 5-fluoro-2-methoxy-phenyl or 5-fluoro-2-hydroxy-phenyl and P is a protecting group.

26. A compound of formula (V) 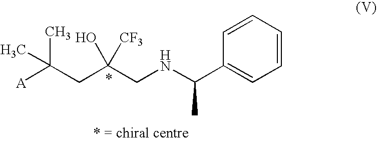
wherein A represents 2,3-dihydro-1-benzofuran-7-yl, 5-fluoro-2-methoxy-phenyl or 5-fluoro-2-hydroxy-phenyl.

27. A method for the treatment of a human subject with an allergic condition, which method comprises administering to said human or animal subject an effective amount of a compound as claimed in claim 1, or a physiologically functional derivative thereof.

28. A method for the treatment of an animal subject with an inflammatory condition, which method comprises administering to said animal subject an effective amount of a compound as claimed in claim 1, or a physiologically functional derivative thereof.

29. A method for the treatment of an animal subject with an allergic condition, which method comprises administering to said animal subject an effective amount of a compound as claimed in claim 1, or a physiologically functional derivative thereof.

30. A method according to claim 14, wherein the skin disease is selected from the group consisting of eczema, psoriasis, allergic dermatitis, neurodermatitis, pruritis and hypersensitivity reactions.

31. A method for the treatment of an animal subject with a skin disease, which method comprises administering to said human or animal subject an effective amount of a compound as claimed in claim 1, or a physiologically functional derivative thereof.

32. A method according to claim 31, wherein the skin disease is selected from the group consisting of eczema, psoriasis, allergic dermatitis, neurodermatitis, pruritis and hypersensitivity reactions.
